(19) United States
(12) Patent Application Publication
(10) Pub. No.: US 2018/0116607 A1
(43) Pub. Date: May 3, 2018

(54) WEARABLE MONITORING DEVICE

(71) Applicant: Garmin Switzerland GmbH, Schaffhausen (CH)

(72) Inventors: Sean Shin Yuan Yu, Overland Park, KS (US); Eric W. Heling, Overland Park, KS (US); Wai C. Lee, Overland Park, KS (US); Adam W. Roush, Prairie Village, KS (US)

(21) Appl. No.: 15/728,140

(22) Filed: Oct. 9, 2017

Related U.S. Application Data
(60) Provisional application No. 62/414,420, filed on Oct. 28, 2016.

(51) Int. Cl. A61B 5/00; A61B 5/024

(52) U.S. Cl. CPC A61B 5/7278; A61B 5/681; A61B 5/746; A61B 5/02427; A61B 5/02438; A61B 5/742

(57) ABSTRACT

A system, method, and device for monitoring a physiological characteristic and/or response of a user includes a wearable monitoring device for displaying a status level of a physiological characteristic and/or response. When attached to or against the user's body, the wearable monitoring device includes an optical signal assembly configured to generate an optical signal based on a reflection or transmission of light from a detected travel of blood and/or pulse wave of the user and received by one or more sensors disposed along the user's extremity. A processor calculates the physiological characteristic and/or response, for example, heart-rate, heart-rate variability, blood pressure, stress intensity level, or energy level of the user, based on the generated signal from the sensor and user data.

### CROSS-REFERENCE TO RELATED APPLICATIONS

The present application claims priority benefit under 35 U.S.C. § 119(e) of U.S. Provisional Patent Application Ser. No. 62/414,420, entitled “Wellness-Related Measurements and Interface,” filed on Oct. 28, 2016, the disclosure of which is expressly incorporated herein by reference in its entirety.

### BACKGROUND

Many health and wellness monitoring systems implement various combinations of mechanical, electrical, and optical devices and components to monitor one or more physiological characteristics associated with the health and/or wellness of an individual. For example, a heart-rate monitor may determine an individual's heart-rate based on an electrocardiogram (ECG) signal generated from electrical signals attained from sensors (e.g., electrodes, conductive pads, etc.) positioned against the individual's body, such as opposite sides of the chest. Other health and wellness monitoring systems may determine an individual's heart-rate based on a photoplethysmogragh (PPG) signal generated by optical sensors (e.g., photodiodes) that receive transmissions or reflections of light output by light-emitting diodes (LEDs) at a location, such as the individual's wrist or a fingertip. Additionally, some heart-rate monitoring systems utilize biometric telemetry and a combination of ECG and PPG signals to determine an individual's heart-rate and/or blood pressure based on travel of blood (pulse wave) between the individual's heart and an extremity of interest, such as a fingertip. However, the combined use of electrical and optical equipment introduces complexities and challenges for accurate measurement of physiological characteristics and, due to the type of medical equipment necessary to generate the ECG and PPG signals and the placement thereof (e.g., torso, wrist, fingertip), these conventional systems impede user movement and are generally considered impractical for use during mobile applications (e.g., walking, running, riding, swimming, rowing, etc.).

The health and wellness monitoring systems described above perform well in a medical environment where the individual is stationary or at rest (e.g., seated or reclined). Unfortunately, such conventional health and wellness monitoring systems are ineffective and/or impracticable for use during daily activities with movement. It is therefore desirable to provide a health and wellness monitoring system for a user that is capable of monitoring and presenting physiological characteristics and/or responses while the user is mobile to enable the user to take timely measures, such taking a break from a current activity or performing relaxation exercises (e.g., controlled breathing).

### SUMMARY

In one aspect of the invention, a wearable monitoring device capable of being worn on a user and determining a stress intensity level of the user, includes a display, a photoplethysmograph (PPG) signal assembly coupled to a memory and configured to generate a PPG signal based on a transmission or reflection of light received from the user's extremity, and a memory configured to store the PPG signal for a first period of time. The wearable monitoring device may include a processor coupled to the display and the memory, the processor configured to determine a first time interval between a first heart-beat and a second heart-beat of the stored PPG signal for the first period of time, determine a second time interval between the second heart-beat and a third heart-beat of the stored PPG signal for the first period of time, determine a heart-rate variability value associated with the first period of time based on the determined first and second time intervals, calculate a stress intensity level based on the determined heart-rate variability value, and control the display to present the calculated stress intensity level.

In another aspect of the invention, a wearable monitoring device may be capable of being worn on or against the body of a user (e.g., on a user's wrist, finger, neck, ear, ankle, neck, torso, etc.) and determining a stress intensity level of the user, includes a display and a photoplethysmograph (PPG) signal assembly coupled to a memory and the display. The PPG signal assembly may be configured to generate a PPG signal based on a transmission or reflection of light received from the user's extremity, the PPG signal assembly including a photodiode and a light-emitting diode (LED), wherein the LED is configured to output light into the user's extremity, wherein the photodiode positioned proximate the LED and configured to detect a pulse wave of the user based on the transmission or reflection of the light from the user's extremity and generate the PPG signal. The wearable monitoring device may also include a memory configured to store the PPG signal for a first period of time and a processor coupled to the display and the memory. The processor may be configured to determine a first time interval between a first heart-beat and a second heart-beat of the stored PPG signal for the first period of time, determine a second time interval between the second heart-beat and a third heart-beat of the stored PPG signal for the first period of time, determine a heart-rate variability value associated with the first period of time based on the determined first and second time intervals, calculate a stress intensity level based on the determined heart-rate variability value, and control the display to present the calculated stress intensity level.

### BRIEF DESCRIPTION OF THE DRAWINGS

FIG. 1 depicts an exemplary process for monitoring a physiological characteristic and/or response of a user as described herein;

FIG. 2A depicts a plot of a PPG signal and a peak-to-peak interval (PPI) for three successive hear beats;

FIG. 2B depicts a plot of a heart rate (beats per minute) determined for a period of one hour;

FIG. 2C depicts a plot of a stress intensity level and a body energy level determined for a period of one hour;

FIG. 3 is a block diagram depicting one embodiment of a health and wellness monitoring system capable of executing the process for monitoring a physiological characteristic and/or response of a user as described herein;

FIGS. 4A and 4B depict views of one embodiment of the health and wellness monitoring system of the present invention as described herein;

FIGS. 5A, 5B, and 5C depict various configurations for placement of the photodiodes of the health and wellness monitoring system of the present invention as described herein;

FIG. 6 depicts an exemplary placement of photodiodes and LEDs of an embodiment of the health and wellness monitoring system of the present invention as described herein;

FIG. 7 is an illustration depicting an example sequence of displays that may be presented on the user interface of the wearable monitoring device as described herein;

FIG. 8 is an illustration depicting various example displays that may be presented on the user interface of the wearable monitoring device as described herein;

FIGS. 9-12 are illustrations depicting an example displays on the user interface of the wearable monitoring device as described herein;

FIG. 13 is an illustration depicting various example displays that may be presented on the user interface of the wearable monitoring device as described herein; and

FIG. 14 is an illustration depicting various example displays providing stress-coping recommendations or relaxation activities that may be presented on the user interface of the wearable monitoring device as described herein.

### DETAILED DESCRIPTION

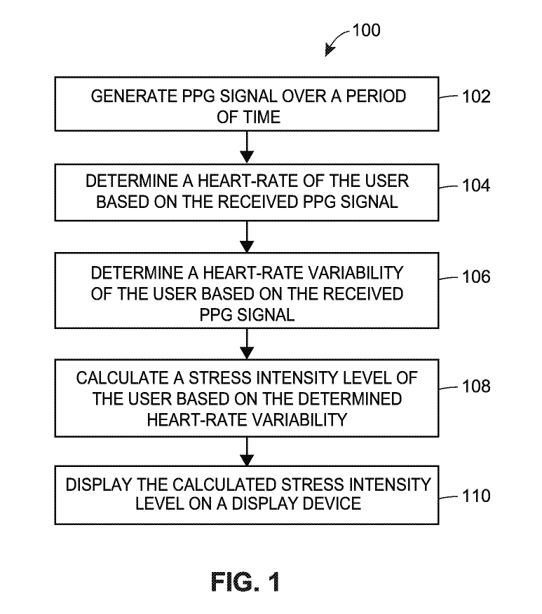

Embodiments of the present invention provide an improved health and wellness monitoring system with the features described herein. FIG. 1 depicts one example method 100 of a health and wellness monitoring system directed to monitoring a physiological characteristic and response of a user. The physiological response may be a physiological characteristic or information associated with the user's physiological characteristic. A physiological response may be determined based on a stored correlation of one or more physiological characteristics. Examples of physiological characteristics include a user's heart-rate ((HR), number of heartbeats per unit of time), heart-rate variability ((HRV) variation in the time interval between successive heartbeats), velocity of a pulse wave ((PWV)—velocity at which an arterial pulse of blood travels through a user's circulatory system), pulse-transit-time ((PTT)—time it takes a pulse wave to travel through a length of the arterial tree). Examples of physiological responses include a stress intensity level and a body energy level.

One embodiment of the health and wellness monitoring system includes a wearable monitoring device having a housing that is attached to (worn on or against the body of) the user. For example, the health and wellness monitoring system may be worn on a user's wrist, finger, neck, ear, ankle, neck, torso, or any other suitable location on the user's body where the components described herein may output light and receive reflections from or transmissions of the light from tissue of the user's skin. The wearable monitoring device may include a processor (e.g., a microcontroller), a memory device, a display device, and a PPG signal assembly, which may be a combination of LEDs and photodiode(s). The PPG signal assembly may include one or more LEDs to output light at a desired wavelength (e.g., green light, red light, infrared light, etc.) and one or more photodiodes to receive reflections or transmissions of the outputted light from an area of interest on the user's body (e.g., wrist, finger, neck, ear, etc.). In embodiments having one photodiode, the photodiode may be positioned on the center of an area of interest. In embodiments having two or more photodiodes, the photodiodes may be positioned apart a distance (less than the width of a housing) along the generally lateral path of blood flow from the shoulder at one end, through the upper and lower arms, and toward the wrist, hand, and fingers at the other end (e.g., along the forearm or wrist). In configurations where the wearable monitoring device includes a watch housing and two photodiodes, the photodiodes may be located on the rear of the housing and the lateral distance between the two photodiodes may be minimal, such as 5-30 mm, and limited by the lateral dimension of the watch housing (e.g., 38-48 mm). In embodiments, the wearable monitoring device may include two housings that enables positioning one photodiode in each housing several inches apart.

Each photodiode samples light reflected from a user's skin tissue proximate thereto and generates a photoplethysmogragh (PPG) signal based on an intensity of light received by the photodiode. Light output from a light-emitting diode passes through one or more layers of the user's skin and portions are reflected out such that some reflected light may be received by the photodiode. and reflected by a detected heart-pulse or pulse-wave (e.g., wave of blood cells) travelling from the heart to the end of the extremity (at block 102). By using the known distance between the two photodiodes and a determined time at which each photodiode detects the pulse wave passing by, the processor may calculate the heart-rate from the PPG signals (at block 104). For example, the pulse wave velocity (PWV) can be calculated by dividing the known lateral distance between the two photodiodes by the amount of time it takes the pulse wave to travel therebetween, i.e., PTT.

In embodiments, the processor may identify individual heart beats associated with values (e.g., peaks) of the PPG signal and a heart-rate variability (HRV) by determining a time period between successive heart beats, as well as fluctuations thereof (block 106). Specifically, the processor may determine a first time interval between a first heart-beat and a second heart-beat of the stored PPG signal for the first period of time. The processor may determine a second time interval between the second heart-beat and a third heart-beat of the stored PPG signal for the first period of time. The processor may store the first time interval and the second time interval in the memory device.

The memory device may also store a table or algorithm providing correlations between of physiological data or user characteristics and a physiological response, such as a stress intensity level and a body energy level. For example, the memory device may include correlations between biological characteristics (e.g., gender, age, weight, weight, etc.) as a reference to estimation for physiological responses. Similarly, the memory device may include correlations between physiological characteristics, such as a heart-rate (HR), a heart-rate variability (HRV), and a blood pressure, and a physiological response (e.g., a stress intensity level, a body energy level, etc.). The physiological data stored in the table or received as inputs to the algorithm may also refer to correlations between additional physiological characteristics, such as a pulse transit time (PTT) and a pulse wave velocity (PWV), hydration status, and a blood oxygen level, for estimating a physiological response (e.g., a stress intensity level, a body energy level, etc.). In embodiments, the table may also provide correlations between fitness characteristics, such as activity information (e.g., motion data) and wellness level (e.g., good health, poor health, etc.), and a physiological response (e.g., a stress intensity level, a body energy level, etc.).

The processor may utilize the stored information to calculate a stress intensity level based on the determined heart-rate variability (HRV) value. For example, a heart-rate-variability (HRV) that is high, based on the correlations stored in the memory device, may be indicative of a lower stress level. On the other hand, a heart-rate-variability(HRV) that is low, based on the correlations stored in the memory device, may be indicative of a higher stress level.

The processor may also determine a PTT or a PWV and utilize the PPT and/or PVW information to determine the stress intensity level or body energy level. For example, the processor may determine, based on correlations stored in the memory device, that fewer variations in the PTT or the PWV may reflect a more consistent, repetitive interval of time between heartbeats and indicative of a higher stress intensity level. Variations in the PTT or the PWV may correspond to variations in the interval of time between heartbeats. In embodiments, the processor may utilize the PPT or PWV information to determine a blood pressure for the user. The memory device may store additional correlations between blood pressure values and a stress intensity level for users having certain biological characteristics (e.g., gender, age, weight, etc.) and fitness characteristics (e.g., active users, inactive users, VO2 Max, etc.).

Additionally, the processor may analyze one or more determined stress intensity levels over a period of time to determine an energy level (body battery) of the user. The sequence of stress intensity levels determined over a period of time, whether, stable, increasing, and/or decreasing, may provide insight to the user's energy level, e.g., stable, energizing, and/or de-energizing.

The measured and/or determined physiological response (e.g., a stress level, an energy level, etc.) and physiological characteristic (at block 108) may be recorded in the memory device and/or displayed on a user interface of the wearable monitoring device (at block 110), and/or transmitted via wired and/or wireless communication to a remote display device or user interface. Similar to the determined first time interval and the second time interval, the calculated or estimated PTTs and PWVs may be stored in the memory device, which may be referenced to one or more physiological characteristics and/or response data stored therewith. For example, the physiological data may include heart-rate variability (HRV), PTTs, or PWVs correlated to stress, blood pressure, hydration status, blood oxygen level, activity (e.g., movement), wellness, etc., and may include other related aspects, such as sex, age, height, weight, etc., of the user and/or a group of individuals with similar or different biological and fitness characteristics.

Embodiments of the present invention may include determination of the user's physiological response, for example, stress intensity level, energy level, and/or a stress recovery measurement. The memory device may include a data structure including a correlation between one or more physiological characteristics and one or more physiological responses. For example, the memory device of the wearable monitoring device may be configured to store a data structure correlating stress intensity level and the determined heart-rate variability (HRV) of a user. Determination of, or changes to a physiological response, which may be related to one or more physiological characteristics of the user (e.g., blood pressure (BP), heart rate (HR), heart-rate variability (HRV), age, physical movement, etc.), may be analyzed and determined by the processor of the wearable monitoring device or by a processor of the health and wellness monitoring system. In embodiments, a processor of a remote computing device (e.g., a server) may receive the information described herein and execute the processes described herein to remotely determine one or more physiological characteristics and one or more physiological responses. The processor may determine heart-rate variability (HRV) based on fluctuations of time between successive heart beats identified in a cardiac component of a PPG signal, which may be generated based on a transmission or reflection of light output by one or more LEDs of the PPG signal assembly after the light has passed through one or more layers of skin proximate to the housing of wearable monitoring device.

In particular, a physiological response may include autonomic nervous system activity of the user, which may be analyzed and determined by the processor to be stressful or relaxing events based on determined changes in a physiological characteristic, e.g., heart rate (HR) and heart-rate variability (HRV) of a cardiac component of a PPG signal. The health and wellness monitoring system may aggregate, and store in the memory device, HR and/or HRV data over a period of time. The processor may retrieve the stored HR and/or HRV data from the memory device and analyze the retrieved data to determine an overall stress level of the user. Blood pressure, which may be determined based on the pulse-transit-time (PTT) or the pulse-wave velocity (PWV), is another physiological characteristic that may be regulated by the autonomic nervous system. In embodiments, the processor may determine and monitor blood pressure over a period of time to determine or change the stress intensity level.

The processor may determine an overall stress level and control the display device to present the overall stress level on a user interface of the health and wellness monitoring system. Overall stress level may be presented on the display device in a textual, numerical, and/or graphical (pictorial) manner. The wearable monitoring device may aggregate a plurality of instantaneous response values (e.g., a stress level, an energy level, etc.) over a period of time to provide trending metrics (e.g., stress trend, energy trend, etc.). For example, stress trending metrics may provide insight or notification to users about increasing, leveled (stable), or decreasing stress levels. The wearable monitoring system may utilize stored historical HR and/or HRV data into consideration when determining stress trending metrics to better determine and predict the progression of a user's current and/or anticipated stress levels. Historical data may include information related to location, activity, time, or personal fitness, such as amount of exercise, recovery time, and sleep metrics. In some embodiments, the historical data stored in the memory device may be determined by the processor or input by the user. Additionally, both stress level and trends may be categorized into different zones based on the magnitudes of each. For example, the wearable monitoring device may express stress level zones as low, medium, and high. Other terms may be used to provide better granularity and understanding of determined stress levels and trends for a user. Similarly, the wearable monitoring device may express trending zones as increasing, neutral, or decreasing.

Additionally, user movement may be considered by the health and wellness monitoring system, including the wearable monitoring device, in the determination and/or utilization of a physiological response and/or characteristic. For example, based on output from a movement sensor (e.g., accelerometer, gyroscope, etc.) within the wearable monitoring device, the processor may be able to determine one or more periods of activity or inactivity based on inertial data for consideration when determining stress levels for the user.

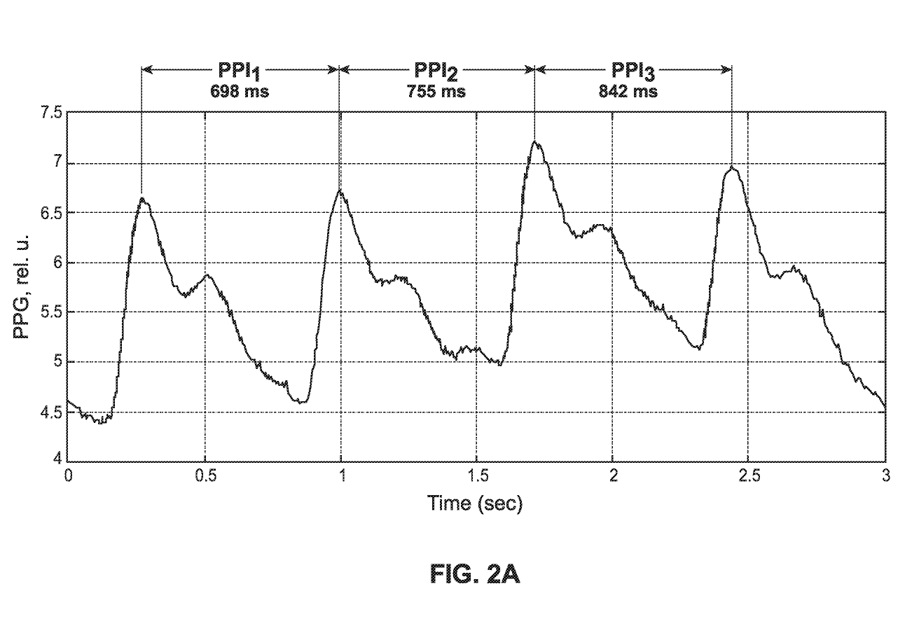

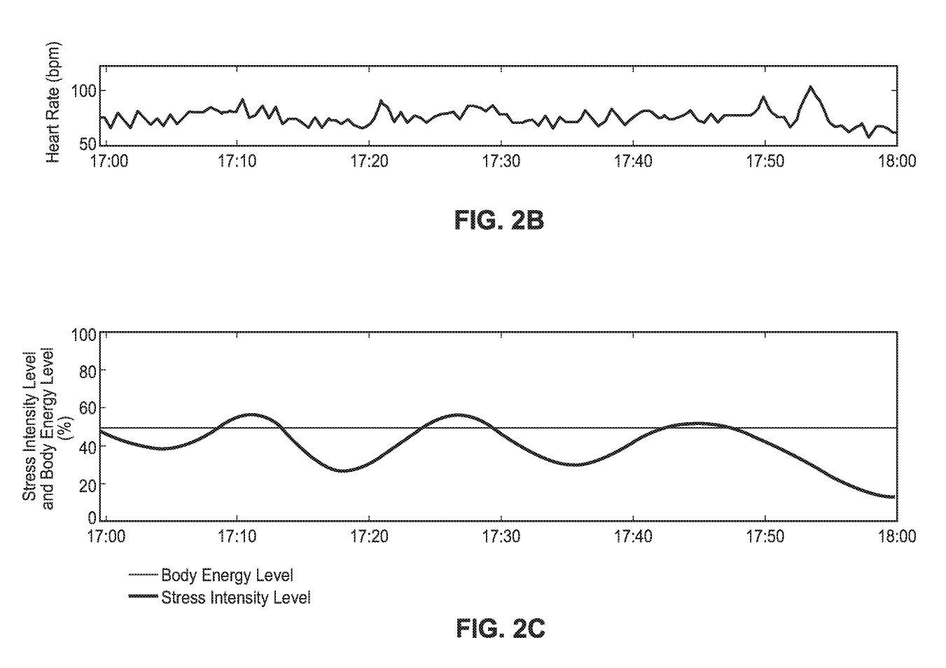

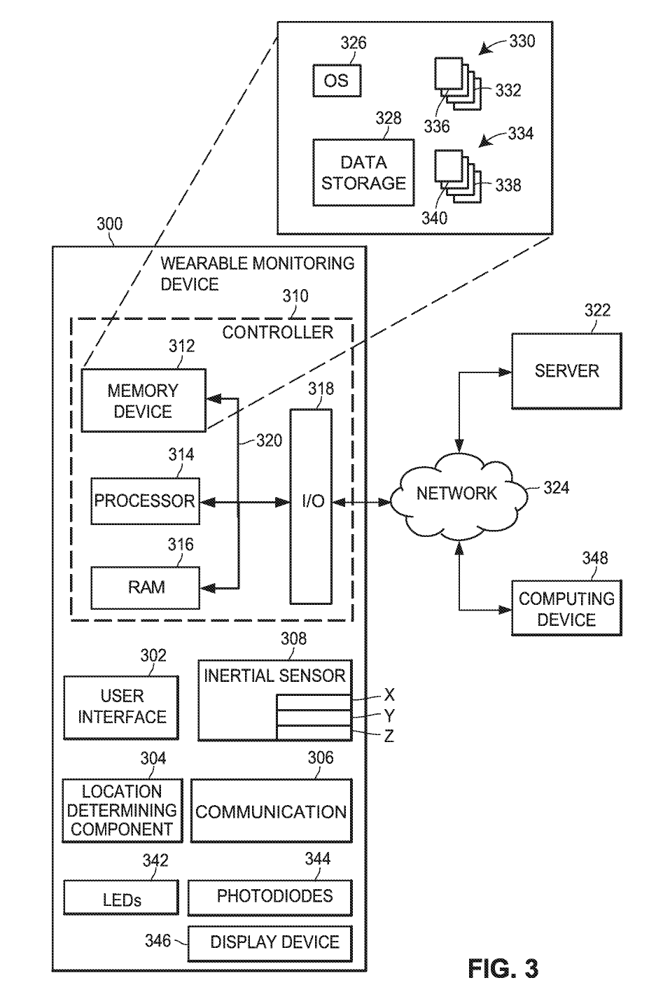

FIGS. 2A, 2B, and 2C are graphs illustrating exemplary relationships among various physiological characteristics and responses (e.g., a stress level, an energy level, etc.) of the user that may be determined or calculated by the wearable monitoring device. Determination of the physiological characteristics and/or responses may include other considerations related to the user, including, but not limited to, current or historical activity/movement levels, age, weight, and health history.

FIG. 2A depicts a PPG signal associated with a plurality of heart beats that occur over a period of three seconds. FIG. 2B depicts a calculated heart-rate (HR), expressed as beats per minute (bpm) over a period of interest. FIG. 2C depicts the calculated stress intensity level and an energy level of the user over the period of interest. Although FIGS. 2B and 2C depict a period of interest of one hour, it is to be understood that the period of interest may be many hours, one or more days or one or more weeks.

As shown in FIG. 2A, an exemplary PPG signal may include four peaks associated with four heart beats occurring within a period of three seconds. The processor may examine the PPG signal for the three seconds (or any relevant period of time) and identify four peaks associated with the four heart beats and three intervals between successive heart beats, which may be referenced as the peak-to-peak interval (PPI). For the example illustrated in FIG. 2A, the processor may identify, based on a time of each peak identified by evaluating continuous values of the PPG signal, a PPI(1), which is an interval between the first and second peaks of the PPG signal, a PPI(2), which is an interval between the second and third peaks of the PPG signal, and a PPI(3), which is an interval between the third and fourth peaks of the PPG signal. The processor may carry out this process “N” number of times and store each of the determined PPI intervals (PPI(1)-PPI(N)) in the memory device.

The processor may retrieve from a memory device the stored PPI intervals corresponding to a period of interest and determine an extent to which the PPI intervals for the period of interest vary or deviate amongst the PPI intervals for the period of interest or between successive PPI intervals. For example, the processor may determine a standard deviation amongst the stored PPI intervals for the period of interest, a mathematical difference (subtraction) between successive PPI intervals, or other techniques to quantify an amount of variation between a set of data. The processor may store this determined variance or deviation between PPI intervals as a determined heart-rate variability (HRV) for the period of interest.

The processor may retrieve from the memory device memory the table providing correlations between a heart-rate variability (HRV) and a stress intensity level for a variety of characteristics (e.g., gender, age, weight, etc.). The processor may utilize the stored information to calculate a stress intensity level based on the determined heart-rate variability (HRV) value for the period of interest. For example, a heart-rate-variability (HRV), based on the correlations stored in the memory device, may be correlated to or indicative of a user's stress level. On the other hand, a heart-rate-variability (HRV) that is low, based on the correlations stored in the memory device, may be indicative of a higher amount or increase in heart-rate variability. Higher heart-rate variability (HRV) is often associated with lower stress intensity levels and periods of relaxation and recovery, which can be observed in FIG. 2C. Conversely, lower heart-rate variability (HRV) is often associated with higher stress intensity levels and periods of stress, which can be observed in FIG. 2C. Peaks of the sinusoidal curve are shown to generally coincide with the instances of increased stress intensity and the trough of the sinusoidal curve generally coincides with the instances of decreasing or reduced stress intensity.

In embodiments, the processor may determine an energy level based at least partially on an aggregated stress intensity level is shown as the sinusoidal curve in FIG. 2C. For example, the processor may analyze one or more determined stress intensity levels over a period of interest to determine a body energy level (body battery) of the user. A sequence of stress intensity levels determined over a period of time to be stable may be utilized by the processor to determine that the user's body energy level is stable. Similarly, a sequence of stress intensity levels determined over a period of time to be increasing may be utilized by the processor to determine that the user's body energy level is energizing. A sequence of stress intensity levels determined over a period of time to be decreasing may be utilized by the processor to determine that the user's body energy level is de-energizing.

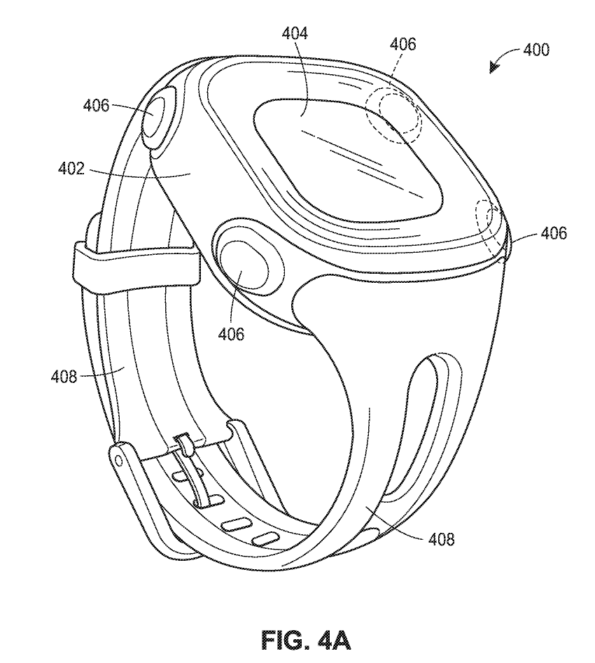

An example embodiment of a wearable monitoring device 300 capable of executing the methods and processes described herein is illustrated in FIG. 3. The device 300 includes a user interface module 302, a location determining component 304 (e.g., a global positioning system (GPS) receiver, assisted-GPS, etc.), a communication module 306, an inertial sensor 308 (e.g., accelerometer, gyroscope, etc.), and a controller 310.

The device 300 may be a general-use wearable and mobile computing device (e.g., a watch, activity band, etc.), a cellular phone, a smartphone, a tablet computer, or a mobile personal computer, capable of monitoring a physiological characteristic and/or response of an individual as described herein. The device 300 may be a thin-client device or terminal that sends processing functions to a server device 322 via a network 324. Communication via the network 324 may include any combination of wired and wireless technology. For example, the network 324 may include a USB cable between the device 300 and a computing device 344 (e.g., smartphone, tablet, laptop, etc.) to facilitate the bi-directional transfer of data between the device 300 and the computing device 344.

The controller 310 may include a memory device 312, a microprocessor (MP) 314, a random-access memory (RAM) 316, and an input/output (I/O) circuitry 318, all of which may be communicatively interconnected via an address/data bus 320. Although the I/O circuitry 318 is depicted in FIG. 3 as a single block, the I/O circuitry 318 may include a number of different types of I/O circuits. The memory device 312 may include an operating system 326, a data storage device 328, a plurality of software applications 330, and/or a plurality of software routines 334. The operating system 326 of memory device 312 may include any of a plurality of mobile platforms, such as the iOS®, Android™, Palm® webOS, Windows® Mobile/Phone, BlackBerry® OS, or Symbian® OS mobile technology platforms, developed by Apple Inc., Google Inc., Palm Inc. (now Hewlett-Packard Company), Microsoft Corporation, Research in Motion (RIM), and Nokia, respectively. The data storage device 328 of program memory 212 may include application data for the plurality of applications 330, routine data for the plurality of routines 334, and other data necessary to interact with the server 322 through the network 324. In particular, the data storage device 328 may include cardiac component data associated with the individual and/or one or more other individuals. The cardiac component data may include one or more compilations of recorded physiological characteristics of the user, including, but not limited to, a PPG signal, a heart rate (HR), a heart-rate variability (HRV), a blood pressure, motion data, a determined distance traveled, a speed of movement, calculated calories burned, body temperature, and the like. In some embodiments, the controller 310 may also include or otherwise be operatively coupled for communication with other data storage mechanisms (e.g., one or more hard disk drives, optical storage drives, solid state storage devices, etc.) that may reside within the device 300 and/or operatively coupled to the network 324 and/or server device 322.

The device 300 also includes a photoplethysmograph (PPG) signal assembly including one or more emitters, such as LEDs 342, and one or more photodiodes 344. The LEDs 342 output visible and/or non-visible light and the one or more photodiodes 344 receive transmissions or reflections of the visible and/or non-visible light. Each LED 342 generates light based on an intensity determined by the processor. For example, LEDs 342 may include any combination of green light-emitting diodes (LEDs), red LEDs, and/or infrared LEDs that may be configured by the processor to emit light into the user's skin.

The device 300 also includes one or more photodiodes 344 capable of receiving transmissions or reflections of visible-light and/or infrared (IR) light output by the LEDs 342 into the user's skin and generating a PPG signal based on the intensity of the reflected light received by each photodiode 344. The light intensity signals generated by the one or more photodiodes 344 may be communicated to the processor. In embodiments, the processor includes an integrated a photometric front end for signal processing and digitization. In other embodiments, the processor is coupled with a photometric front end. The photometric front end may include filters for the light intensity signals and analog-to-digital converters to digitize the light intensity signals into PPG signals including a cardiac signal component associated with the user's heartbeat.

Typically, when the device 300 is worn against the user's body (e.g., wrist, fingertip, ear, etc.), the one or more LEDs 342 are positioned against the user's skin to emit light into the user's skin and the one or more photodiodes 344 are positioned near the LEDs 342 to receive light emitted by the one or more emitters after transmission through or reflection from the user's skin. The processor 314 of device 300 may receive a PPG signal based on a light intensity signal output by one or more photodiodes 344 based on an intensity of light after transmission of the light through or reflection from the user's skin that has been received by the photodiodes 344.

In both the transmitted and reflected uses, the intensity of measured light may be modulated by the cardiac cycle due to variation in tissue blood perfusion during the cardiac cycle. In activity environments, the intensity of measured light may also be strongly influenced by many other factors, including, but not limited to, static and/or variable ambient light intensity, body motion at measurement location, static and/or variable sensor pressure on the skin, motion of the sensor relative to the body at the measurement location, breathing, and/or light barriers (e.g., hair, opaque skin layers, sweat, etc.). Relative to these sources, the cardiac cycle component of the PPG signal can be very weak, for example, by one or more orders of magnitude.

The location determining component 304 generally determines a current geolocation of the device 300 and may process a first electronic signal, such as radio frequency (RF) electronic signals, from a global navigation satellite system (GNSS) such as the global positioning system (GPS) primarily used in the United States, the GLONASS system primarily used in the Soviet Union, or the Galileo system primarily used in Europe. The location determining component 304 may include satellite navigation receivers, processors, controllers, other computing devices, or combinations thereof, and memory. The location determining component 304 may be in electronic communication with an antenna 30 that may wirelessly receive an electronic signal from one or more of the previously-mentioned satellite systems and provide the first electronic signal to location determining component 304. The location determining component 304 may process the electronic signal, which includes data and information, from which geographic information such as the current geolocation is determined. The current geolocation may include geographic coordinates, such as the latitude and longitude, of the current geographic location of the device 300. The location determining component 304 may communicate the current geolocation to the processor 314. Generally, the location determining component 304 is capable of determining continuous position, velocity, time, and direction (heading) information.

In some embodiments, the inertial sensor 308 may incorporate one or more accelerometers positioned to determine the acceleration and direction of movement of the device 300. The accelerometer may determine magnitudes of acceleration in an X-axis, a Y-axis, and a Z-axis to measure the acceleration and direction of movement of the device 300 in each respective direction (or plane). It will be appreciated by those of ordinary skill in the art that a three-dimensional vector describing a movement of the device 300 through three-dimensional space can be established by combining the outputs of the X-axis, Y-axis, and Z-axis accelerometers using known methods. Single and multiple axis models of the inertial sensor 308 are capable of detecting magnitude and direction of acceleration as a vector quantity, and may be used to sense orientation and/or coordinate acceleration of the user.

The PPG signal assembly (including LEDs 342 and photodiodes 344), location determining component 304, and the inertial sensors 308 may be referred to collectively as the “sensors” of the device 300. It is also to be appreciated that additional location determining components 304 and/or inertial sensor(s) 308 may be operatively coupled to the device 300. The device 300 may also include or be coupled to a microphone incorporated with the user interface module 302 and used to receive voice inputs from the user while the device 300 monitors a physiological characteristic and/or response of the user determines physiological information based on the cardiac signal.

The communication module 306 may enable device 300 to communicate with the computing device 344 and/or the server device 322 via any suitable wired or wireless communication protocol independently or using I/O circuitry 318. The wired or wireless network 324 may include a wireless telephony network (e.g., GSM, CDMA, LTE, etc.), one or more standard of the Institute of Electrical and Electronics Engineers (IEEE), such as 802.11 or 802.16 (Wi-Max) standards, Wi-Fi standards promulgated by the Wi-Fi Alliance, Bluetooth standards promulgated by the Bluetooth Special Interest Group, a near field communication standard (e.g., ISO/IEC 18092, standards provided by the NFC Forum, etc.), and so on. Wired communications are also contemplated such as through universal serial bus (USB), Ethernet, serial connections, and so forth.

The device 300 may be configured to communicate via one or more networks 324 with a cellular provider and an Internet provider to receive mobile phone service and various content, respectively. Content may represent a variety of different content, examples of which include, but are not limited to: map data, which may include route information; web pages; services; music; photographs; video; email service; instant messaging; device drivers; real-time and/or historical weather data; instruction updates; and so forth.

The user interface 302 of the device 300 may include a “soft” keyboard that is presented on the display device 346 of the device 300, an external hardware keyboard communicating via a wired or a wireless connection (e.g., a Bluetooth keyboard), and/or an external mouse, or any other suitable user-input device or component. As described earlier, the user interface 302 may also include or communicate with a microphone capable of receiving voice input from a vehicle operator as well as a display device 346 having a touch input.

With reference to the controller 310, it should be understood that controller 310 may include multiple microprocessors 314, multiple RAMs 216 and multiple memory devices 312. The controller 310 may implement the RAM 316 and the memory devices 312 as semiconductor memories, magnetically readable memories, and/or optically readable memories, for example. The one or more processors 314 may be adapted and configured to execute any of the plurality of software applications 330 and/or any of the plurality of software routines 334 residing in the memory device 312, in addition to other software applications. One of the plurality of applications 330 may be a client application 332 that may be implemented as a series of machine-readable instructions for performing the various functions associated with implementing the performance monitoring system as well as receiving information at, displaying information on, and transmitting information from the device 300. The client application 332 may function to implement a system wherein the front-end components communicate and cooperate with back-end components as described above. The client application 332 may include machine-readable instructions for implementing the user interface 302 to allow a user to input commands to, and receive information from, the device 300. One of the plurality of applications 330 may be a native web browser 336, such as Apple's Safari®, Google Android™ mobile web browser, Microsoft Internet Explorer® for Mobile, Opera Mobile™, that may be implemented as a series of machine-readable instructions for receiving, interpreting, and displaying web page information from the server device 322 or other back-end components while also receiving inputs from the device 300. Another application of the plurality of applications 230 may include an embedded web browser 342 that may be implemented as a series of machine-readable instructions for receiving, interpreting, and displaying web page information from the server device 322 or other back-end components within the client application 332.

The client applications 330 or routines 334 may include an accelerometer routine 338 that determines the acceleration and direction of movements of the device 300, which correlate to the acceleration, direction, and movement of the user. The accelerometer routine 338 may receive and process data from the inertial sensor 308 to determine one or more vectors describing the motion of the user for use with the client application 332. In some embodiments where the inertial sensor 308 includes an accelerometer having X-axis, Y-axis, and Z-axis accelerometers, the accelerometer routine 338 may combine the data from each accelerometer to establish the vectors describing the motion of the user through three-dimensional space. In some embodiments, the accelerometer routine 338 may use data pertaining to less than three axes.

The client applications 330 or routines 334 may further include a velocity routine 340 that coordinates with the location determining component 304 to determine or obtain velocity and direction information for use with one or more of the plurality of applications, such as the client application 332, or for use with other routines.

The user may also launch or initiate any other suitable user interface application (e.g., the native web browser 336, or any other one of the plurality of software applications 230) to access the server device 322 to implement the monitoring process. Additionally, the user may launch the client application 332 from the device 300 to access the server device 322 to implement the monitoring process.

After the above-described data has been gathered or determined by the sensors of the device 300 and stored in memory device 312, the device 300 may transmit information associated with the PPG signal (cardiac component), peak-to-peak interval (PPI), heart rate (HR), heart-rate variability (HRV), motion data (acceleration information), location information, stress intensity level, and body energy level of the user to computing device 344 and server device 322 for storage and additional processing. For example, in embodiments where the device 300 is a thin-client device, the computing device 344 or the server device 322 may perform one or more processing functions remotely that may otherwise be performed by the device 300. In such embodiments, the computing device 344 or server device 322 may include a number of software applications capable of receiving user information gathered by the sensors to be used in determining a physiological response (e.g., a stress level, an energy level, etc.) of the user. For example, the device 300 may gather information from its sensors as described herein, but instead of using the information locally, the device 300 may send the information to the computing device 344 or the server device 322 for remote processing. The computing device 344 or the server device 322 may perform the analysis of the gathered user information to determine a stress level or a body energy level of the user as described herein. The server device 322 may also transmit information associated with the physiological response, such as a stress level, an energy level, of the user. For example, the information may be sent to a computing device 344 or the server device 322 and include a request for analysis, where the information determined by the computing device 344 or the server device 322 is returned to device 300.

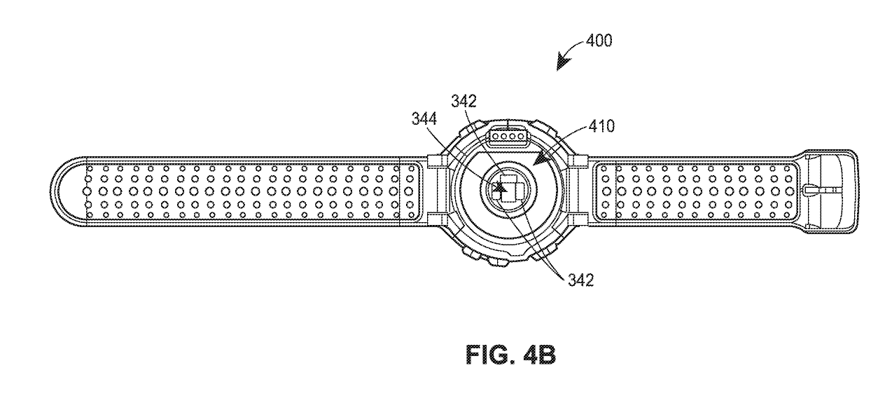

The disclosed techniques and described embodiments may be implemented in a wearable monitoring device having a housing implemented as a watch, a mobile phone, a hand-held portable computer, a tablet computer, a personal digital assistant, a multimedia device, a media player, a game device, or any combination thereof. The wearable monitoring device may include a processor configured for performing other activities. FIGS. 4A and 4B illustrate views of one example embodiment of the device 400 of the monitoring system 300 for monitoring physiological responses and/or characteristics as described above.

The device 400 may be configured in a variety of ways to determine and provide wellness information, including one or more cardiac components, as well as navigation functionality to the user of the device 400. The device 400 includes a housing or a case 402 configured to substantially enclose various components of the device 400. The housing 402 may be formed from a lightweight and impact-resistant material such as plastic, nylon, or combinations thereof, for example. The housing 402 may be formed from a conductive material, a non-conductive material, and combinations thereof. The housing 402 may include one or more gaskets, e.g., a seal, to make it substantially waterproof and/or water resistant. The housing 402 may include a location for a battery and/or another power source for powering one or more components of the device 400. The housing 402 may be a singular piece or may include multiple sections.

The device 400 includes a display device and a user interface 404 similar to user interface 302 and display device 346. The display device 404 may include a liquid crystal display (LCD), a thin film transistor (TFT), a light-emitting diode (LED), a light-emitting polymer (LEP), and/or a polymer light-emitting diode (PLED). The display device 404 may be capable of presenting text, graphical, and/or pictorial information. The display device 404 may be backlit such that it may be viewed in the dark or other low-light environments. One example embodiment of the display device 404 is a 100 pixel by 64 pixel film compensated super-twisted nematic display (FSTN) including a bright white light-emitting diode (LED) backlight. The display device 404 may include a transparent lens that covers and/or protects components of the device 400. The display device 404 may be provided with a touch screen to receive input (e.g., data, commands, etc.) from a user. For example, a user may operate the device 400 by touching the touch screen and/or by performing gestures on the screen. In some embodiments, the touch screen may be a capacitive touch screen, a resistive touch screen, an infrared touch screen, combinations thereof, and the like. The device 400 may further include one or more input/output (I/O) devices (e.g., a keypad, buttons, a wireless input device, a thumbwheel input device, etc.). The I/O devices may include one or more audio I/O devices, such as a microphone, speakers, and alike. Additionally, user input may be provided from movement of the housing 402, for example, an inertial sensor(s), e.g., accelerometer, may be used to identify vertical, horizontal, and/or angular movement of the housing 402.

In accordance with one or more embodiments of the present disclosure, the user interface 404 may include one or more control buttons 406. As illustrated in FIG. 4A, four control button 406 are associated with, e.g., adjacent, the housing 302. While FIG. 4A illustrates four control buttons 406 associated with the housing 402, it is to be understood that the device 300 may include more or less control buttons 406. Each control button 406 is configured to generally control a function of the device 400. Functions of the device 300 may be associated with a location determining component and/or a performance monitoring component. Functions of the device 400 may include, but are not limited to, displaying a current geographic location of the device 400, mapping a location on the display 404, locating a desired location and displaying the desired location on the display 404, and presenting information based on a physiological characteristic (e.g., heart-rate, heart-rate variability, blood pressure etc.) or a physiological response (e.g., stress level, body energy level, etc.) of the individual.

The device 400 also includes an PPG signal assembly 410, as shown in FIG. 4B, including one or more emitters (e.g., LEDs 342) of visible and/or non-visible light and one or more receivers (e.g., photodiodes 344) of visible and/or non-visible light that generate a light intensity signal based on the received reflection of light.

The device 400 includes a means for attaching 408, e.g., a strap, that enables the device 400 to be worn by the user. In particular, when the device is worn by the user, one or more LEDs and one or more photodiodes may be securely placed against the skin of a user. The strap 308 is coupled to and/or integrated with the housing 402 and may be removably secured to the housing 402 via attachment of securing elements to corresponding connecting elements. Some examples of securing elements and/or connecting elements include, but are not limited to, hooks, latches, clamps, snaps, and the like. The strap 408 may be made of a lightweight and resilient thermoplastic elastomer and/or a fabric, for example, such that the strap 408 may encircle a portion of a user without discomfort while securing the device 400 to the user. The strap 408 may be configured to attach to various portions of a user, such as a user's leg, waist, wrist, forearm, upper arm, and/or torso.

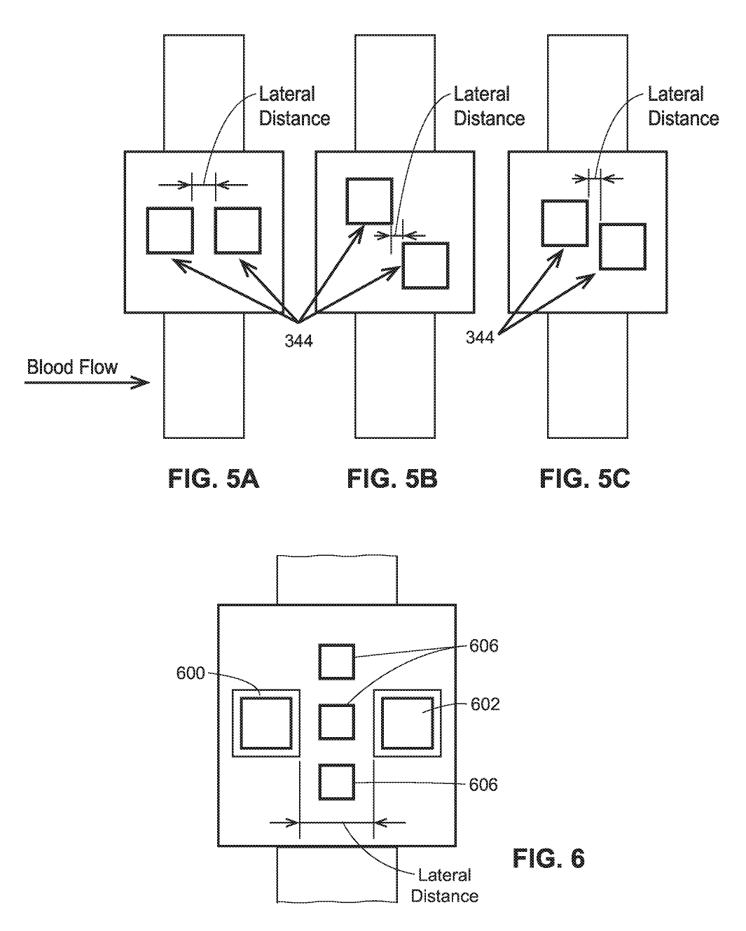

In embodiments, the wearable monitoring device includes a plurality of photodiodes 344 and a plurality of LEDs 342. FIGS. 5A, 5B, and 5C depict different configurations of two photodiodes 344 for positioning on a portion of a user's extremity or limb, such as the user's neck, lower arm, wrist, ankle, or torso. In accordance with the present invention, two or more photodiodes 344 are positioned on the user's skin tissue along an arterial path that is substantially parallel with a longitudinal axis of the user's extremity. The two photodiodes 344 are horizontally aligned and separated by a lateral distance that is substantially parallel with the longitudinal axis of the extremity, e.g., forearm, when the photodiodes 344 are attached to a user. Each photodiode 344 independently samples the adjacent skin tissue to detect a pulse wave as it travels from the heart to the end of the extremity. Although the two photodiodes 344 are horizontally positioned, the two photodiodes may be vertically offset with respect to each other, which may not adversely affect detection of the pulse wave as it travels along the limb and subsequent calculation of the physiological characteristic. That is, one photodiode 344 may be positioned closer to the ulna bone and the other photodiode 344 may be positioned closer to the radius bone (see FIGS. 5B and 5C), and visa-versa. As discussed herein, the processor of the wearable monitoring device is configured to utilize a known (stored) lateral distance, e.g., horizontal separation, between the two photodiodes 344 to determine a PTT and/or a PWV, and subsequently, a physiological characteristic, such as heart rate, heart-rate variability, blood pressure, of the user by performing the techniques disclosed herein. The processor may utilize the physiological characteristic(s) to determine a physiological response, such as stress level and body energy level.

The wearable monitoring device includes at least one LED 342 positioned sufficiently near the two photodiodes 344 to enable the photodiodes 344 to operatively receive reflected light that was emitted from the at least one LED 342 and reflected from the user's skin tissue or transmitted through the user's soft tissue. In some embodiments, a plurality of LEDs 342 may be positioned around each and/or both photodiodes 344 such that the photodiodes 344 receive reflected or transmitted light emitted from the plurality of LEDs 342.

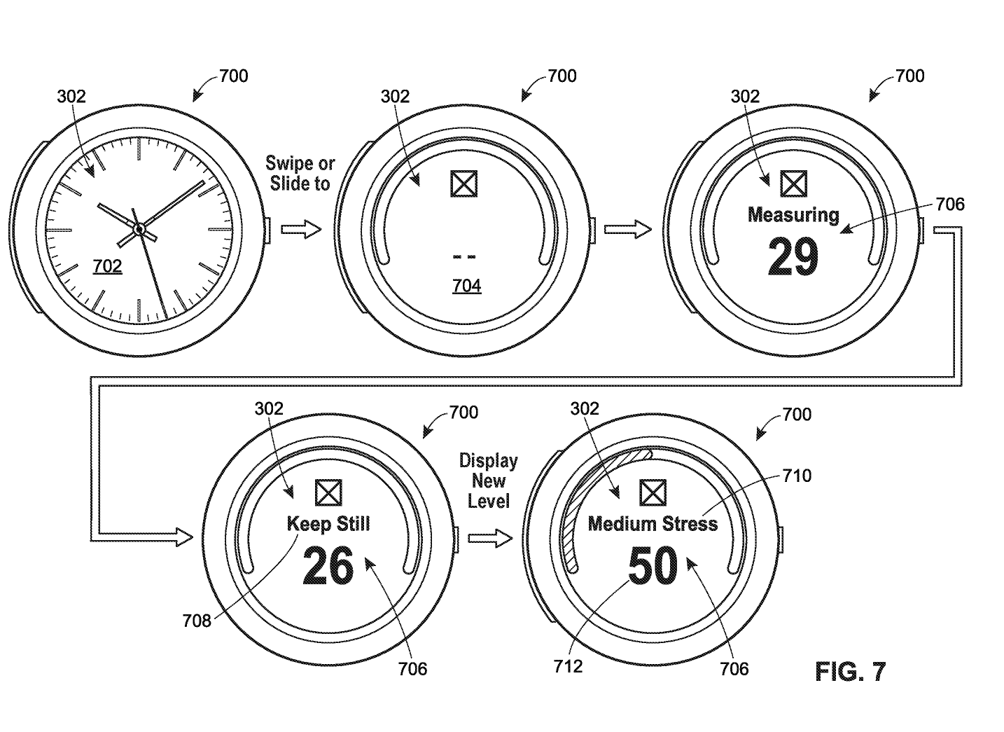

For example, in FIG. 6, the wearable monitoring device may include two photodiodes 600, 602 aligned horizontally and a plurality of LEDs 606 vertically positioned between the two photodiodes 600, 602. The plurality of LEDs 606 may extend between the user's ulna and radius bones such that the light sensed by each photodiode 600, 602 is output by the shared LEDs 606. In another embodiment, the wearable monitoring device may include two or more photodiodes 600, 602 and a combination of one or more shared LEDs 606 positioned between the two photodiodes 600, 602 and producing light sensed by the two or more photodiodes 600, 602 and/or one or more unshared LEDs 606 that produce light that may be concentrated at the side of each photodiode 600 farther from the other photodiode 602.

When the two or more photodiodes 600, 606 are positioned close to each other, a higher sampling rate may be beneficial for each photodiode to generate the PPG signals to enable the processor to differentiate the peak of the pulse wave at the first photodiode 600 from the peak of the pulse wave at the second photodiode 606, which will occur shortly after the pulse wave passes by the first photodiode 600. At a sufficiently close distance, the second photodiode 60 may begin to detect (sense) the rise of the pulse wave before it has completely passed the first photodiode.

A high sampling rate for the photodiodes 600, 606 also enables higher resolution of the PPG signal to be sampled. This in turn enables the processor to identify and determine the PPG signal peaks with better precision, which enables the peak detection and cross-correlation algorithms to be more accurate. Each photodiode may generate a PPG signal by sampling a detected pulse wave at a high sampling frequency, such as 50-2,000 Hz and provide the PPG signal to the processor and/or a memory device of the wearable monitoring device. The memory device may be included within the wearable monitoring device and/or may be remote to the wearable monitoring device.

In embodiments of the present invention, the processor may determine a stress intensity level and provide stress recovery measurement features. As detailed herein, determination of, or changes in, determined physiological characteristics of the user, such as blood pressure (BP), heart-rate (HR), heart-rate variability (HRV), may be analyzed by the processor of the device 400 to determine physiological responses, such as a stress intensity level for the wearer of the wearable monitoring device 300. In one embodiment, the processor 314 may determine a heart-rate variability (HRV) based on fluctuations of peak-to-peak intervals (PPI) corresponding to changes in a duration of time between successive heart beats identified in a cardiac component of a PPG signal. The PPG signal may be generated by a photodiode 344 based on the intensity of light reflections or transmissions received by the photodiode 344 of light output by the one or more LEDs 342 after the light has passed through the user's skin proximate to the housing of wearable monitoring device 300.

Additionally, autonomic nervous system activity may be analyzed and determined by the processor to be stressful or relaxing events based on determined changes in HR, HRV, and/or blood pressure. Physiological data (e.g., BP, HR, HRV, etc.) may be determined by the processor and aggregated into a memory device 312 over a period of time. The processor 314 may retrieve the stored physiological data from the memory device 312 and analyze the data to determine an overall stress intensity level of the user. The overall stress intensity level may be presented on a user interface 302 provided on the display device 346 of the wearable monitoring device 300. The processor 314 may also determine and monitor the user's physiological characteristics and physiological response, such as stress intensity level, over a period of time to provide current physiological characteristics and physiological response on the user interface 302. Overall stress intensity level may be presented on the user interface 302 in a textual, numerical, and/or graphical (pictorial) manner.

In some embodiments, the wearable monitoring device 300 may retrieve physiological data (e.g., BP, HR, HRV, etc.) from the memory device 312 and determine body energy level information. The processor 314 may retrieve physiological data that may have been acquired during one or more periods of time to determine a stress intensity level for each period of time, as well as an overall body energy level for the user.

For example, the processor 314 may aggregate multiple stress intensity values to provide stress trending information. The stress trending information may include metrics providing insight to the user about increasing, leveled (stable, neutral), or decreasing stress levels and body energy level. The processor 314 may take historical data into consideration when determining stress trending metrics to better determine and predict the progression of a user's current and anticipated stress levels and body energy levels. Historical data may include information related to location, activity, time, or personal fitness, such as amount of exercise, recovery time, sleep metrics, etc. The historical data may be detected by the processor and/or input by the user. Both stress intensity levels and body energy levels may be categorized into different zones based on the magnitudes of each. For example, the processor 314 may express stress level zones as low, medium, and high. In embodiments, resting, low, medium, and high stress level zones may be associated with stress intensity levels of 0-25, 26-50, 51-75, 76-100, respectively. Other terms may be used to provide better granularity and understanding of determined stress levels and trends for the user.

The health and wellness monitoring system may also include an accelerometer for consideration during monitoring of the physiological characteristics and physiological responses. With the availability of motion data provided by the accelerometer, the processor 314 may identify the type of physical activity of the user and adjust the sampling cycle or peak-to-peak intervals (PPI) for determining a user's heart beat, heart-rate variability (HRV), and blood pressure, accordingly.

The processor 314 may also control the PPG signal assembly to determine, store and retrieve heart beat, heart-rate variability, and blood pressure measurements at pre-determined rate during a specified activity or operating mode. For instance, if processor 314 determines that the user is engaged in an activity of riding a bike, the processor 314 may control the PPG signal assembly to enable determination of a heart beat, heart-rate variability (HRV), and blood pressure measurement every 30 seconds during the ride. Conversely, if processor 314 determines that the user is engaged in a sedentary activity, the processor 314 may control the PPG signal assembly to enable determination of a heart beat, heart-rate variability (HRV), and blood pressure measurement less frequently, such as every 2 minutes. In such examples, the processor 314 may control the PPG signal assembly by increasing or decreasing the rate at which one or more LEDs 342 output light and/or increasing or decreasing the rate at which one or more photodiodes 344 generate a PPG signal based on the intensity of received light reflections from the user's skin or transmissions through the user's soft tissues.

In some embodiments, the processor 314 may receive from user interface 302 an input from a user indicating the user's desire for the wearable monitoring device 300 to provide enhanced monitoring. For instance, the user may select a “Watch Me Closely” menu option to initiate an associated operating mode. The processor 314 may control the PPG signal assembly to increase the rate at which one or more LEDs 342 output light and increasing the rate at which one or more photodiodes 344 generate a PPG signal based on the intensity of received light reflections or transmissions from the user's skin. The processor 314 may subsequently determine and store in memory device 312 physiological data at a higher rate than the normal operation one corresponding a determined heart beat, heart-rate variability (HRV), and blood pressure at an increased rate during the enhanced monitoring period as well.

Processor 314 of the wearable monitoring device may also determine periods of activity or inactivity based on motion data output from the inertial sensor 308, which may include an accelerometer or gyroscope, for consideration in the determination of a stress intensity level or a body energy level of the user. In particular, the processor 314 may retrieve from the memory device 312 the table providing correlations between of physiological data or user characteristics and a physiological response, such as a stress intensity level and a body energy level. The processor 314 may take movement of the user into account when determining a stress intensity level.

In embodiments, processor 314 may prevent or suspend the display of the stress intensity level during periods of excessive movement to avoid presenting inaccurate and/or inconsistent results to the user because the stress intensity level may be affected by the movement. For example, the processor 314 may suspend display of the stress intensity level until the user reduces physical movement, attains a stable physiological characteristic (e.g., reduced HR, HRV, etc.), or a predetermined amount of time elapses, whereupon the processor 314 may allow and/or resume display of the stress intensity level on the user interface 302. In embodiments, during such periods of suspended display, the wearable monitoring device 300 may provide instructions, requests, and/or commands to the user advising of the prevented display and/or recommending user actions.

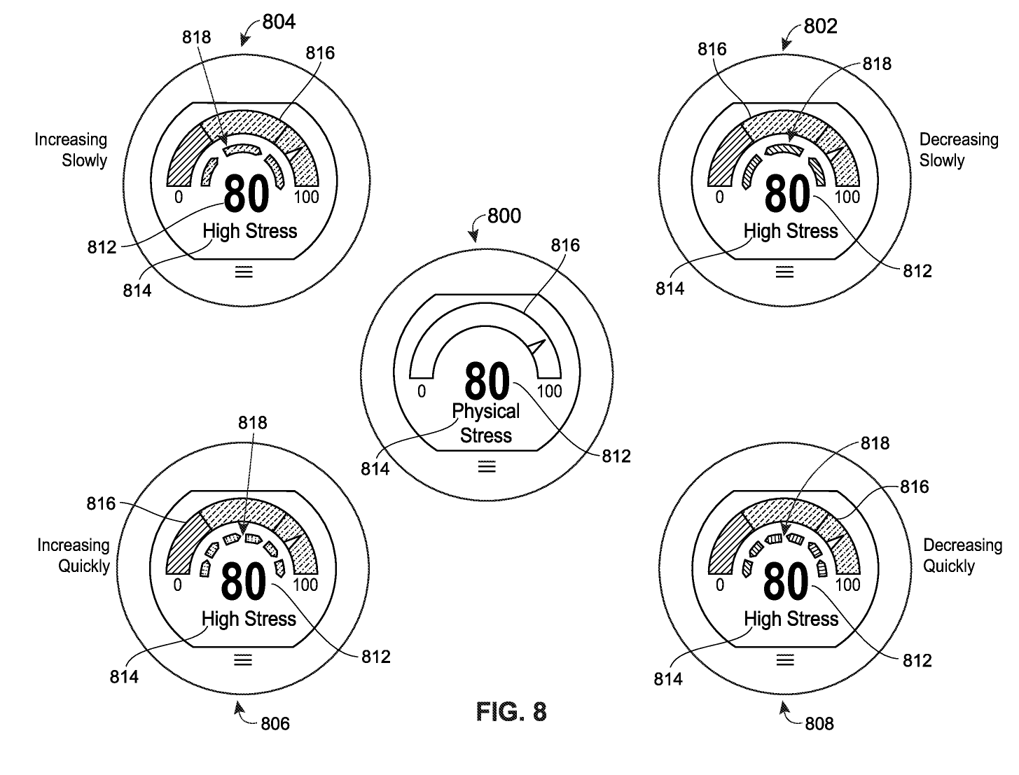

FIG. 7 shows a sequence of exemplary user interfaces 302 that may be presented on the display device 304 of the wearable monitoring device 300 (device 700) to communicate determined stress levels and/or wellness trends in accordance with embodiments of the invention. In the exemplary user interface 302 shown in FIG. 7, a watch face 702 is displayed on the user interface of the device 700. To initiate a display of the stress intensity level, the user may interact with the user interface 302 of device 700. For example, the user may swipe across the touch screen area of the user interface 302, e.g., watch face dial, to initiate measurement and display of the stress intensity level on display device 304. Additionally, or alternately, the user may speak a command (audible speech) to the device 700 or activate a button or switch to initiate measurement and display of the user's stress intensity level by the processor 314. In response to detection of the user gesture, an initial display 704 is displayed on the user interface 302 of the mobile device prior to activating the monitoring of a physiological characteristic and a physiological response, such as a stress intensity level, of the user. Another interim display that may appear on the user interface prior to, during, or after monitoring of the physiological characteristic and a physiological response may include presenting one or more indicators 706 associated with measured and/or calculated physiological responses or characteristics of the user of the device 700. The indicator may provide a current status or value of the physiological characteristic. The indicator 706 may be textual, numerical, and/or graphical (pictorial) in any manner. For example, the indicator 706 includes a digital timer displaying an amount of time that remains or has lapsed during the monitoring process. Additionally, the indicator 706 may include text describing the status of the monitoring functionality of the device. Other indicators, such as graphics, may also be presented. It is to be understood that instructions 708 may be displayed on the user interface of the device 700. One example instruction 708 includes text, “Keep Still,” displayed along with the indicator 706. Ultimately, the device 700 displays one or more indicators 706 representative of the determined stress intensity level of the user. For example, a textual indicator 710 provides a stress zone, e.g., “Medium Stress,” associated with the calculated stress intensity level, that may be provided using a numerical indicator 712 e.g., “50.” It is to be understood that device 700 is not limited to the indicators shown in FIG. 7, and that any combination of indicators may be displayed on the user interface 302 as desired.

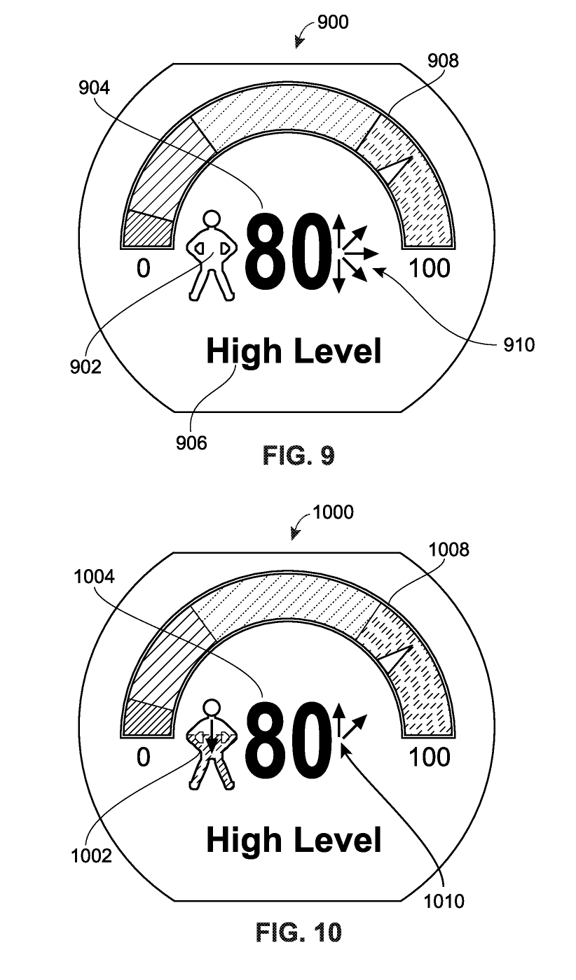

FIG. 8 includes illustrations of other example indicators presented by the processor 314 to provide a trend associated with calculated physiological characteristics or responses of the user of the wearable monitoring device 300. The display device on user interface 800 (center of FIG. 8) includes a numerical indicator 812, a textual stress zone indicator 814, and/or a graphical indicator 816 for the calculated physiological response and/or characteristic. The graphical indicator 816 may include three distinct regions corresponding to three stress zones (low, medium and high). The numerical indicator 812, which may be attained from the calculated numerical value stored in memory, also corresponds, and/or is a counterpart of, the textual stress zone indicator 814. Similarly, the graphical indicator 816 includes a graphic that corresponds to, and/or is a counterpart of, the numerical indicator 812 and the textual stress zone indicator 814.

In user interface 800, the pointer points to the right-most portion of the spectrum which may normally be in a color, e.g., red, to indicate higher calculated values. However, to denote that the user is engaged in a physical activity, which may affect the determined stress intensity level value, the color of the entire graphical indicator 816, e.g., spectrum, may be different than normally shown and to alert the user that the stress level value may be affected by the physical activity. For example, the graphic indicator 816 may be shaded blue. Additionally, a large numeric indicator 812 (“80”) is shown to indicate that the user's physical intensity level is essentially equivalent to where the pointer is pointing on the spectrum (range of 0 to 100). A pointer points to a portion of the graphical indicator 816, e.g., spectrum, that is towards the right side of the spectrum to indicate that the user's determined stress level is high. Additionally, the exemplary numerical indicator 812 “80” is presented to indicate that the user's determined stress intensity level is “80” within a range of 0 to 100. Additionally, a textual stress zone indicator 814 that corresponds to the numerical indicator 812 is also displayed, wherein the text “High Stress” is presented to indicate the stress zone.

In embodiments, the processor 314 may provide the alert using additional techniques. For instance, the device 300 may include a speaker and the processor 314 may utilize the speaker to provide the stress alert. Similarly, the device 300 may include a vibrating (haptic) element and the processor 314 may utilize the vibrating element to provide the stress alert.

In embodiments, the processor 314 may provide an alert when a determined physiological response (e.g., a stress level, an energy level, etc.) exceeds a predetermined threshold stored in memory device 312. For instance, memory device 312 may store a plurality of values associated with zones of a physiological response, such as a “resting” stress level zone, a “low” stress level zone, a “medium” stress level zone, and a “high” stress level zone, and the processor may control the display device 304, a speaker, or a vibrating (haptic) element to notify the user when the determined physiological response is transitioning between two or more zones consecutively or over a period of time to enable the user to proactively reduce the stress level by taking adequate precautionary measures. In embodiments, the processor 314 may determine an average physiological response for a user and control the display device 304, a speaker, or a vibrating (haptic) element to notify the user once the physiological response exceeds a triggering threshold stored in memory. For example, the processor 314 may control the display device 304, a speaker, or a vibrating (haptic) element to notify the user when a determined physiological response exceeds the average by a standard deviation of the determined physiological response stored in the memory device 312.

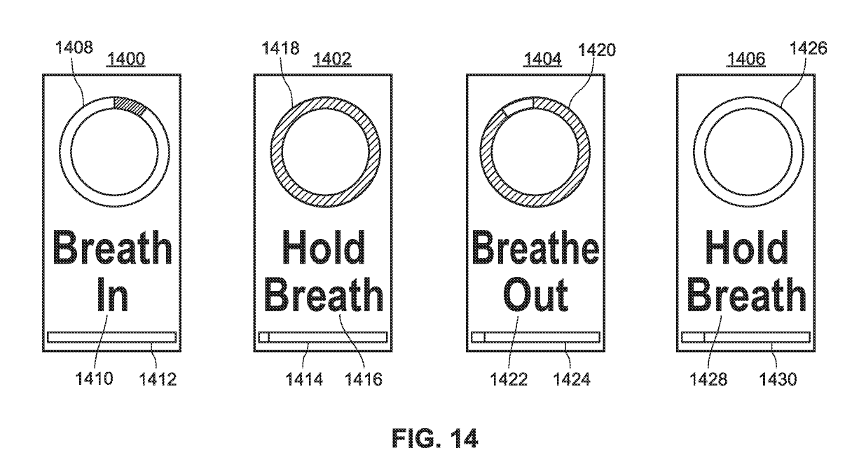

In embodiments, the processor 314 may present a user interface 302 notifying the user of an elevated stress intensity level (a call to action) to enable the user to proactively reduce the stress level, such as by performing relaxation activities, such as mild physical exercise, or relaxation exercises, such as breathing exercises, as shown in FIG. 14. In embodiments, the user interface 302 may provide an option for the user to acknowledge and temporarily dismiss the notification. The processor 314 may continue monitoring the stress intensity level to determine whether the user took adequate precautionary measures to reduce the stress level. The processor 314 may store in the memory device 312 the determined stress intensity level for which the notification was provided on the user interface 302, the subsequent actions of the user in response to notification, and the subsequent stress level intensities.

In embodiments, the processor 314 may utilize the stored information to determine whether to provide on the user interface a stress intensity level notification for similar stress level intensities. For example, for users determined to take adequate precautionary measures after being notified of a physiological response (e.g., a stress level, an energy level, etc.), the processor 314 may acknowledge the threshold as valid and continue to provide all notices to the user based on the learned experience. Alternatively, for users determined not to take adequate precautionary measures after being notified of a physiological response (e.g., a stress level, an energy level, etc.), the processor 314 may acknowledge the rejection and only provide high-priority notices to the user. For instance, the processor 314 may control the display device 304, a speaker, or a vibrating (haptic) element to notify the user of a determined physiological response (e.g., a stress level, an energy level, etc.) escalates from a “medium” stress level zone to a “high” stress level zone. In embodiments, similarly, if the processor 314 typically provides a notification when a determined physiological response exceeds an average value by a standard deviation of the determined physiological response stored in the memory device 312, the processor may control the display device 304, a speaker, or a vibrating (haptic) element to notify the user when a determined physiological response (e.g., a stress level, an energy level, etc.) exceeds the average value by two standard deviations.

In embodiments, the processor 314 may present a trend indicator 818 on the display device 304 based on the recent change (i.e., change over a short window before the measurement time) in physiological characteristics. The trend indicator 818 may be numerical, textual, and/or graphical in manner. In some embodiments, one or more segments (e.g., bars, arrows) of the trend indicator 818 may be illuminated in one or more colors sequentially or at once to communicate the rate at which a determined physiological characteristic or physiological response, such as stress level, is changing and the direction (increasing or decreasing) of the change. In FIG. 8, the trend indicator 818 includes a line disposed between the numerical indicator 812 and the graphical indicator 816. The trend indicator 818 includes a plurality of segments and provides an indication to the user of the trend of physiological response, such as a stress intensity level and a body energy level. The trend indicator 818 may include one or more colors, which may be animated during display to indicate the trend (e.g., increasing, decreasing, stable, etc.) and a direction, rate, intensity, or duration, of the trend (e.g., slowly or quickly). It is to be understood that although the figures are shown in gray-scale, portions of the display may be shown in color to further indicate an aspect of the displayed information. Traditional (and/or non-traditional) colors may be utilized to denote awareness of preferred or non-preferred trends. For example, the color red may denote a negative or detrimental trend of the physiological response and/or characteristic, the colors yellow and orange may denote a cautious trend, and the color green may denote a positive or improving trend. Further, the presentation of the one or more segments may be animated or unanimated (static).

As shown in user interface 804, the trend indicator 818 includes a line (e.g., curvilinear, arc) with a plurality of segments or bars (3), which are presented under the graphical indicator 816, e.g., spectrum, to indicate the rate at which the user's stress level is changing. In this example, the presented segments(s) may be illuminated (e.g., in red color) to indicate that the change in determined stress level is increasing. Alternatively, the presented segment(s) may be illuminated in another color, such as green, to indicate that the change in determined stress intensity level is decreasing.

In addition, the segments of the trend indicator 818 line may be sized to denote the rate of the trend. For example, long or longer segments may denote a slow rate of change, and short or shorter segments may denote a fast rate of change. In embodiments, the trend indicator 818 and/or its color/shading may be animated as well. For example, segments or portions of the trend indicator 818 may blink at a slow or slower rate to indicate a slower trend, while a fast or faster blink rate may indicate a faster trend.

In user interface 802, the trend indicator 818 includes a line (e.g., curvilinear, arc) with a plurality of segments or bars (3), which are presented under the graphical indicator 816, e.g., spectrum, to indicate the rate at which the user's stress level is changing at slow rate. In this example, three long green bars are presented to indicate that the user's stress level is decreasing at a slow rate.

In user interface 806, the arrow points to the right side portion of the spectrum to indicate that the user's determined stress level is high and a corresponding textual stress zone indicator 814, “High Stress,” is presented under the numeric indicator 812 identifying a determined stress intensity level of “80” within a range of 0 to 100. In this example, trend indicator 818 includes a line (e.g., curvilinear, arc) with a plurality of line segments (6) of the trend indicator 818 presented below the spectrum 816 to indicate that the user's determined stress level is changing (increasing) at a fast(er) rate than as illustrated in user interface 804. The rate of the change in the stress intensity level may be denoted by the number of segments and the shape/size of the one or more segments pointing towards a higher end of the stress level portion of the spectrum.

In user interface 808, the arrow points to the right side portion of the spectrum 816 to indicate that the user's determined stress level is high and a corresponding textual stress zone indicator 814 “High Stress,” is presented under the numeric stress level indicator 812 identifying a determined stress intensity level of “80” within a range of 0 to 100. In this example, in comparison to user interfaces 802 and 806, trend indicator 818 includes a line (e.g., curvilinear, arc) with a plurality of line segments (6) of the trend indicator 818 presented below the spectrum 816 to indicate that the user's determined stress level is changing (decreasing) at a fast(er) rate than as illustrated in user interface 802. In embodiments, the six segments are presented in a different color (e.g., green), and a shape (e.g., arrow) pointing towards a lower end of the stress level portion of the spectrum 816 to denote that the trend of the stress level is decreasing quickly. As stated previously, animation of the trend indicator 818 may be utilized, wherein faster blinking portions denote faster moving trends and slower blinking portions denote slower blinking trends.

In embodiments, the processor 314 may present information determined to help reduce the user's current stress level (a “call to action”) when a predetermined threshold for stress is determined. The “call to action” may include, but is not limited to, haptic and/or audible notifications; verbiage, icon(s), color(s), and/or animation(s) presented on display device 306 of the wearable monitoring device 300. Information that may be presented on user interface 302 to reduce a user's current stress level may include stress-coping recommendations (e.g., breathing exercises) and/or relaxation activities (e.g., mild physical exercise). Calls to action information and/or reminders thereof may time out after a certain period of time and may be removed with greater intensity or shorter interval for calls to action and/or reminders going forward.

In some embodiments of the invention, processor 314 may determine physiological response of a body energy level based on physiological characteristics (e.g., heart rate, heart-rate variability (HRV), blood pressure, etc.) and physiological responses, such as a determined stress intensity level. For example, processor 314 may determine a stress intensity level and identify positive behavior, such as moments or relaxation and sessions of physical exercise, and accumulate and combine that information throughout the day. For example, the device 300 may calculate a body energy level of the user based on a comparison of the calculated stress intensity level associated with the first period of time and the calculated stress intensity level associated with the second period of time, and a movement of the user during the corresponding first and second time periods. Sleep and other relaxing activities, such as naps, recreation, and so on, may also be included for body energy level considerations.

In embodiments, the processor 314 may evaluate recovery and depletion of the body energy level based on changes in a determined heart-rate variability (HRV) and an amount and an intensity of physical activities. Time spent accumulating and consuming body energy may be measured and tracked by the processor 314 to determine and indicate a duration of body energy recovery and depletion. For example, the wearable monitoring device 300 may determine recharging and discharging states based on a determined trend of the body energy level. The rates of recharging and discharging may be calculated based on the combination of incremental changes in energy level and changes in instantaneous stress and/or relaxing response. The discharge rate of energy level may include, but is not limited to, intensity and number of stressful events, loading of physical activities, and/or intensity of physical loadings. The recharge rate of energy level may be calculated based on heart-rate variability and may be combined with some other contextual information, such as location, time of day, activities previously engaged in, and so on.

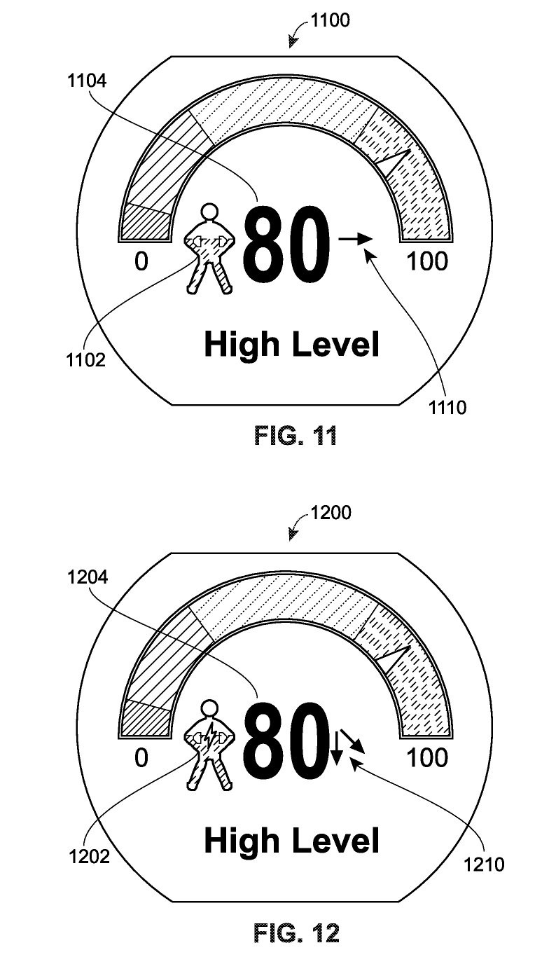

FIG. 9 shows an example user interface 900 displayed on display device 304 of the wearable monitoring device 300 and including a body energy indicator 902 denoting that the display is representative of a determined body energy level of the user. The body energy indicator 902 may include text and/or a graphic, an example of which is shown as a silhouette or outline of a human body. An energy level indicator 902 is representative of the current energy level the user. The energy level indicator may be numerical 904, textual 906, and/or graphical 908 in manner. The energy level numerical indicator 904, which may be attained from the calculated numerical value stored in memory, also corresponds, and/or is a counterpart of, the energy level textual indicator 906. Similarly, the graphical indicator 908 includes a graphic that corresponds to, and/or is a counterpart of, the numerical indicator 904 and the energy level textual indicator 906. In user interface 900, the determined energy level is displayed by the energy level textual indicator 906 (e.g., “High Level” for high level of body energy). A counterpart numerical indicator 904 displays the energy level to the user as a number and/or a portion of 100 (e.g., “80” for 80/100 or 80 percent total energy level).

In embodiments, the processor 314 may present information on the user interface 302 corresponding to a trend in a determined body energy level. An energy trend indicator 910 may also be included in the display. The energy trend indicator 910 may be numerical, textual, and/or graphical in manner. One example of the energy trend indicator 910 is displayed as an arrow of appropriate color and direction to indicate the trend of energy (e.g., accumulating/replenishing or consuming/discharging). In some embodiments, the arrow may communicate the rate at which a determined body energy level is changing and the direction (increasing or decreasing) of the change.

FIG. 10 shows an example user interface 1000 displayed on display of wearable monitoring device in accordance with embodiments of the invention. User interface 1000 illustrates an example embodiment of integration of both the stress monitoring and the body energy level tracking functions. The graphical arch-shape gauge 1008 indicates a graphic that corresponds to, and/or is a counterpart of, the numerical indicator and the energy level textual indicator, the body energy indicator 1002 graphically indicates a current body energy level. In FIG. 10, user interface 1000 indicates that the user is in a state of discharging body energy. That is, the outline or silhouette of the body energy indicator 1002 is approximately 80% filled (with a color, e.g., green) to illustrate the current approximate energy level (e.g., 80 out of 100), and there is a downward pointing arrow (e.g., red) within the silhouette or outline of the body energy indicator 1002 to indicate that energy is discharging as the trend of change in current body energy level. In the discharging state, a colored (e.g., red or orange) upward pointing arrow 1010 may be displayed for the trending indicator of stress level 80 to indicate ongoing stress when the instantaneous physiological response is determined to be increasing.

FIG. 11 shows an example user interface 1100 displayed on display of the wearable monitoring device 300 in accordance with embodiments of the invention. User interface 1100 indicates that the user's body energy level is in a stable or neutral state (e.g., neither discharging nor accumulating energy) by the body energy indicator 1102 without an arrow and that the user's stress intensity level is in a stable or neutral state (neither increasing nor decreasing) by the horizontally positioned arrow 1110. Specifically, the body energy indicator 1102 is approximately 80% filled (e.g., in green) to illustrate the current approximate energy level (e.g., 80 out of 100), but there is no arrow or the horizontal arrow to indicate neither discharge nor accumulation of energy to communicate a stable state. In the neutral state, a yellow horizontal arrow may be displayed for the trending indicator of stress level when the instantaneous physiological response is determined to be stable.

FIG. 12 shows an example user interface displayed on the display of the wearable monitoring device 300 in accordance with embodiments of the invention. The user interface 1200 indicates that the user is in a state of recharging (increasing) body energy. The body energy indicator 1202 is approximately 80% filled (e.g., in green) to illustrate the current approximate energy level (e.g., 80 out of 100), and there is a lightning bolt disposed within the silhouette or outline of the body energy indicator 1202 to indicate that energy is recharging. The illustrated lightning bolt can also be replaced by other indicators that imply an increase (e.g., an upward arrow). In the recharging state, a downward pointing colored arrow (e.g., shades of green) may be displayed for a stress level trending indicator 1210 to indicate recovery when the instantaneous physiological response is determined to be decreasing.

In configurations, the wearable monitoring device 300 may pair with another device, such as a computing device 348 (e.g., smartphone, tablet, laptop, etc.), to facilitate the bi-directional transfer of data between the device 300 and the computing device 348. Such communication functionality may enable the device 300 to sync data or otherwise allow the user to interact with the wearable monitoring device 300 and/or review information and data provided by the wearable monitoring device 300. An application (app) may be stored and executed on the wearable monitoring device 300 and the computing device 348 to provide this user experience. In embodiments, the wearable monitoring device may sync automatically on a certain interval, automatically after a certain amount of data is available to sync, upon request by the user, or upon any other desired event or threshold.

The wearable monitoring device and/or its accompanying app may generate smart reminders for the user to check a physiological characteristic, such as heart rate (HR), heart-rate variability (HRV), or blood pressure, and physiological responses, such as a stress intensity level or a body energy level. In embodiments, the notification provided on the display device 346 may include a timer and a current zone of the stress intensity level (low, medium or high stress), a current zone of a body energy level (low, medium or high body energy level).

The wearable monitoring device 300 may provide notifications or reminders based on a set time interval or determined physiological characteristics or physiological responses. For example, the processor 314 may provide a notification once it determines that the user's physiological response, such as stress intensity level or body energy level, exceeds or falls below a predetermined threshold in the table stored in the memory device 312. Similarly, the processor 314 may provide a notification once it determines that the user's physiological response, such as stress intensity level or body energy level, exceeds or falls below a predetermined threshold and the user is determined to be inactive based on motion data (e.g., accelerometer data, determined steps, etc.) falling below a predetermined movement threshold. The processor 314 may receive a user input to postpone the reminder or the processor 314 may postpone the reminder until additional physiological data may be collected. The smart reminder may also be turned off completely or set manually by the user as desired. Additionally or alternatively, push notifications may be used to remind the user to check or adjust user behavior based on a determined physiological characteristic (e.g., heart rate (HR), heart-rate variability (HRV), blood pressure, etc.) or physiological response (e.g., stress intensity level, body energy level, etc.).

The processor 314 may also control the user interface 302 to present rewards, achievements, or other encouragements to provide progress or successful measurements and collection of physiological data. For example, the wearable monitoring device 300 may provide on the display device 346 streak tracking (e.g., keeping track of how many times a user has hit a particular target, such as a certain number of consecutive BP measurements within a desired range or a certain number of consecutive days of taking BP measurements). The wearable monitoring device may congratulate the user or reward the user, for example, sending notifications to connected third parties, such as friends or relatives, which may prompt them to congratulate or otherwise recognize the user.

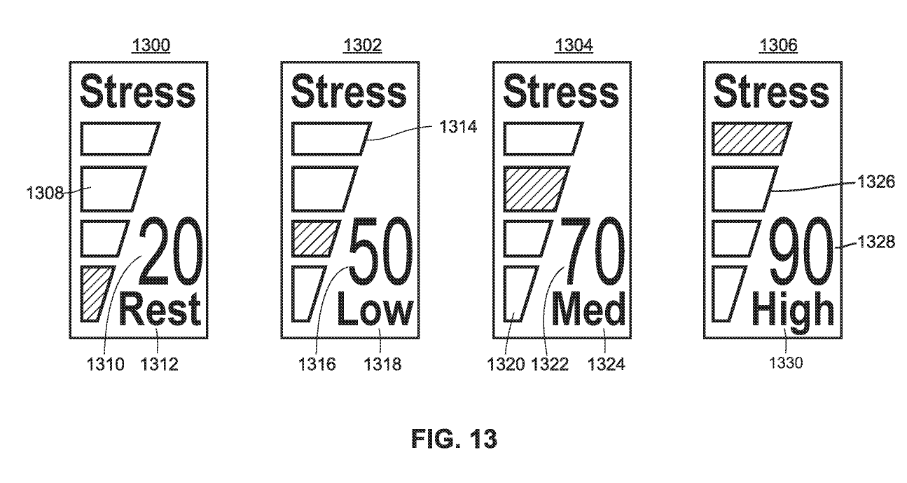

In embodiments, the processor 314 of the wearable monitoring device 300 may control the display device 346 to present a user interface including a determined stress level and/or wellness trends in accordance with embodiments of the invention. The user interface may provide a current status or value of a physiological characteristic or physiological response. The user interface may include textual, numerical, and/or graphical (pictorial) content. In the exemplary user interfaces 1300-1306, as shown in FIG. 13, may present a bar-type graphic element 1308, 1314, 1320, 1326. The user interfaces 1300-1306 may also present a numerical indicator 1310, 1316, 1322, 1332 of the determined stress intensity level. The user interfaces 1300-1306 may also present a textual stress zone indication 1312, 1318, 1324, 1330 are presented to communicate a determined stress level (e.g., resting, Low stress, Medium stress, High stress, etc.). The bar-type graphic element 1308, 1314, 1320, 1326 may include multiple segments that are illuminated individually, as illustrated in FIG. 13, or as a group (to give the effect of the bar-type graphic element 1308 filling as a determined stress intensity level increases). The user may interact with the user interface 1300 of device 300 to select information to be presented. It is to be understood that device 300 is not limited to the indicators shown in FIG. 13, and that any combination of indicators may be displayed on the user interfaces 1300-1306 as desired.

In embodiments, the processor 314 of the wearable monitoring device 300 may control the display device 346 to present a user interface that includes stress-coping recommendations and information to assist a user reduce a user's current stress level by performing relaxation exercises, such as breathing exercises, or relaxation activities, such as mild physical exercise. For example, as shown in FIG. 14, the processor 314 may present a user interface 1400-1406 including a series of steps that are described using an annular graphic element 1408, 1418, 1420, 1426 that is filled (shaded) corresponding to a passing timer for each step or series of steps as well as a textual recommendation 1410, 1416, 1422, 1428. In embodiment, the processor 314 may determine that the series of steps, such as the four steps depicted in FIG. 14, may retrieve information stored in the memory device 312 to determine a duration for which the series of steps may be performed. For example, the processor 314 may provide this information on user interfaces 1400-1406 using a bar 1412, 1414, 1424, 1430 that has a first portion that is filled (shaded) corresponding to a time that has passed and a second portion that is unfilled (unshaded) corresponding to a remaining time to complete the determined duration for performing the series of steps.

The applications and benefits of the systems, methods, and techniques described herein are not limited to only the above examples. Many other applications and benefits are possible by using the systems, methods, and techniques described herein. Thus, many modifications and variations may be made in the techniques and structures described and illustrated herein without departing from the spirit and scope of the present invention. Accordingly, it should be understood that the methods and apparatus described herein are illustrative only and are not limiting upon the scope of the invention.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Also, unless a claim element is defined by reciting the word “means” and a function without the recital of any structure, it is not intended that the scope of any claim element be interpreted based on the application of 35 U.S.C. § 112(f) and/or pre-AIA 35 U.S.C. § 112, sixth paragraph.

Moreover, although the foregoing text sets forth a detailed description of numerous different embodiments, it should be understood that the scope of the patent is defined by the words of the claims set forth at the end of this patent. The detailed description is to be construed as exemplary only and does not describe every possible embodiment because describing every possible embodiment would be impractical, if not impossible. Numerous alternative embodiments could be implemented, using either current technology or technology developed after the filing date of this patent, which would still fall within the scope of the claims.

What is claimed:

1. A wearable monitoring device capable of being attached to a user and determining a stress intensity level of the user, the device comprising:
a display;
a photoplethysmograph (PPG) signal assembly coupled to a memory and configured to generate a PPG signal based on a reflection or transmission of light received from the user's body;
a memory configured to store the PPG signal for a first period of time;
a processor coupled to the display and the memory, the processor configured to:
determine a first time interval between a first heart-beat and a second heart-beat of the stored PPG signal for the first period of time;
determine a second time interval between the second heart-beat and a third heart-beat of the stored PPG signal for the first period of time;
determine a heart-rate variability value associated with the first period of time based on the determined first and second time intervals;
calculate a stress intensity level based on the determined heart-rate variability value; and
control the display to present the calculated stress intensity level.

2. The wearable monitoring device of claim 1, wherein the PPG signal assembly comprises a light-emitting diode (LED) and a photodiode positioned proximate the LED, wherein the LED is configured to output light into the user's body, and wherein the photodiode is configured to generate the PPG signal based on a reflection or transmission of the outputted light from the user's body.

3. The wearable monitoring device of claim 1, wherein the processor further configured to determine a heart rate of the user based on the stored PPG signal and calculate the stress intensity level of the user based on the determined heart rate of the user.

4. The wearable monitoring device of claim 1, wherein the display of the calculated stress intensity level includes a textual indicator, a numerical indicator, and/or a graphical indicator.

5. The wearable monitoring device of claim 1, wherein the processor further configured to calculate a trend of the calculated stress intensity level.

6. The wearable monitoring device of claim 5, wherein the display includes a trend indicator associated with the calculated trend of the calculated stress intensity level.

7. The wearable monitoring device of claim 1, wherein the memory is further configured to store a plurality of stress level zones, and wherein processor is further configured to:
determine whether the calculated stress intensity level is transitioning from a first stress level zone to a second stress level zone, and
control the display to present an alert.

8. The wearable monitoring device of claim 1, wherein the memory is further configured to store a plurality of calculated stress intensity levels, and wherein processor is further configured to:
determine an average value and a standard deviation for the stored stress intensity levels,
determine the triggering threshold based on the average value and the standard deviation,
determine whether a calculated stress intensity level exceeds the triggering level, and
control the display to present an alert.

9. The wearable monitoring device of claim 1, further comprising:
a housing at least partially containing the PPG signal assembly, the processor, the display, and the memory; and
a movement sensor at least partially contained by the housing and coupled to the PPG signal assembly, the movement sensor configured to detect movement of the housing, wherein the processor is further configured to control the display to cease presentation of the calculated stress intensity level once the detected movement exceeds a threshold level stored in the memory.

10. The wearable monitoring device of claim 1, further comprising a vibrating element, wherein the processor is further configured to control the vibrating element to output an alert based on the calculated stress intensity level.

11. The wearable monitoring device of claim 1, wherein the memory is further configured to store a data structure correlating the stress intensity level and the determined heart-rate variability of the user.

12. The wearable monitoring device of claim 1, wherein the memory is further configured to store the PPG signal for a second period of time, and wherein the processor is further configured to:
determine a first time interval between a first heart beat and a second heartbeat of the stored PPG signal for the second period of time;
determine a second time interval between the second beat and a third heartbeat of the stored PPG signal for the second period of time;
determine heart-rate variability value associated with the second period of time based on the determined first and second time intervals for the second period of time;
calculate a stress level intensity based on the determined heart-rate variability value associated with the second period of time.

13. The wearable monitoring device of claim 12, wherein the processor is further configured to:
calculate a trend of stress intensity level of the user based on a comparison of the calculated stress intensity level associated with the first period of time and the calculated stress intensity level associated with the second period of time; and
control the display to present the calculated trend of stress intensity level.

14. The wearable monitoring device of claim 12, wherein the processor is further configured to:
calculate an energy level of the user based on a comparison of the calculated stress intensity level associated with the first period of time and the calculated stress intensity level associated with the second period of time, and movement of the user during the first and second time periods; and
control the display to present the calculated energy level of the user.

15. A wearable monitoring device capable of being attached to a user and determining a stress intensity level of the user, the device comprising:
a display;
a photoplethysmograph (PPG) signal assembly coupled to a memory and the display, the PPG signal assembly configured to generate a PPG signal based on a reflection or transmission of light received from the user's body, the PPG signal assembly including:
a photodiode and a light-emitting diode (LED), wherein the LED configured to output light into the user's extremity, wherein the photodiode positioned proximate the LED and configured to detect a pulse wave of the user based on the reflection or transmission of the light from the user's body and generate the PPG signal;
a memory configured to store the PPG signal for a first period of time;
a processor coupled to the display and the memory, the processor configured to:
determine a first time interval between a first heart-beat and a second heart-beat of the stored PPG signal for the first period of time;
determine a second time interval between the second heart-beat and a third heart-beat of the stored PPG signal for the first period of time;
determine a heart-rate variability value associated with the first period of time based on the determined first and second time intervals;
calculate a stress intensity level based on the determined heart-rate variability value; and
control the display to present the calculated stress intensity level.

16. The wearable monitoring device of claim 15, wherein the processor further configured to determine a heart rate of the user based on the stored PPG signal and calculate the stress intensity level of the user based on the determined heart rate of the user.

17. The wearable monitoring device of claim 15, wherein the display of the calculated stress intensity level includes a textual indicator, a numerical indicator, and/or a graphical indicator.

18. The wearable monitoring device of claim 15, wherein the processor further configured to calculate a trend of the calculated stress intensity level.

19. The wearable monitoring device of claim 15, further comprising:
a housing at least partially containing the PPG signal assembly, the processor, the display, and the memory; and
a movement sensor at least partially contained by the housing and coupled to the PPG signal assembly, the movement sensor configured to detect movement of the housing, wherein the processor is further configured to control the display to cease presentation of the calculated stress intensity level once the detected movement exceeds a threshold level.

20. The wearable monitoring device of claim 15, wherein the memory is further configured to store a data structure correlating the stress intensity level and the determined heart-rate variability of the user.
